(19) United States
(12) Patent Application Publication
(10) Pub. No.: US 2025/0064385 A1
(43) Pub. Date: Feb. 27, 2025

(54) WEARABLE DEVICES, SYSTEMS, AND RELATED METHODS

(71) Applicant: GLOBUS MEDICAL, INC., AUDUBON, PA (US)

(72) Inventors: Patrick Murray, Collegeville, PA (US); Caelan Allen, Ambler, PA (US); Sarah Wolverton, Phoenixville, PA (US); George Yacoub, Lansdale, PA (US)

(21) Appl. No.: 18/459,485

(22) Filed: Sep. 1, 2023

Related U.S. Application Data
(63) Continuation of application No. 18/455,786, filed on Aug. 25, 2023.

(51) Int. Cl. A61B 5/00

(52) U.S. Cl. CPC A61B 5/4566; A61B 5/0053; A61B 5/6805

(57) ABSTRACT

Devices, systems, and methods thereof for assessing patient specific data. A system for preoperatively assessing a patient may include a wearable device, such as a vest having a plurality of segmented compartments configured to apply force(s) onto the patient's spine, a pump connected to the vest and configured to inflate the compartments of the vest, and a computer for receiving patient specific data from the vest. The force(s) and resulting displacement to the spine may be measured and used to calculate the stiffness of the spine, range of motion, flexibility, or other biomechanical information for the patient.

### CROSS REFERENCE TO RELATED APPLICATION(S)

This application is a continuation of U.S. patent application Ser. No. 18/455,786, filed on Aug. 25, 2023, which is hereby incorporated by reference in its entirety for all purposes.

### FIELD OF THE INVENTION

The present disclosure relates to preoperative and postoperative systems for obtaining and assessing patient data, and more particularly, to wearable devices configured to obtain patient specific data, such as spine stiffness, range of motion, and flexibility.

### BACKGROUND OF THE INVENTION

Spine stiffness and range of motion are important parameters that surgeons may consider when making a surgical plan to correct spinal deformities. The degree of these parameters may dictate which correction maneuvers may be performed by the surgeon and what amount of correction may be expected. Current methods of preoperatively assessing patient spine stiffness and motion parameters may include side bending or fulcrum bending tests, push-prone tests, traction, push-traction, and/or flexion/extension imaging. Unfortunately, these methods rely solely on displacement and do not consider any force information. In some instances, the tests also rely on compliance by the patient to follow instructions. In addition, most of these assessments are highly limited in the amount of data that may be captured and may require a large amount of interpolation or extrapolation of the data.

In order to assess the true mechanical stiffness and flexibility of a patient's spine, it may be advantageous to have a system that simultaneously measures both spinal displacement and the force causing it over a given period of time. Other important preoperative metrics may include heart rate, level of exercise, hydration, and other pertinent medical information. As such, there exists a need for a system that obtains and assesses preoperative and/or postoperative data so the surgeon may have a better understanding of the patient's specific spinal biomechanics to allow for better patient outcomes.

### SUMMARY OF THE INVENTION

To meet this and other needs, and in view of its purposes, the present application provides devices, systems, and methods for obtaining, measuring, and collecting patient specific data, such as spine stiffness, range of motion, flexibility, etc. In particular, an external spinal biomechanics system may be used to collect patient data, including preoperative and/or postoperative data, in real time. In one embodiment, the system may include a wearable device, such as a vest, which the patient may temporarily wear prior to surgery to accurately collect patient specific data, such as spine stiffness. The wearable vest may include a plurality of segmented compartments strategically positioned around the patient's torso. Each compartment of the vest may be configured to apply a force onto an area of the patient to create a displacement in the patient's spine. The force and displacement may be measured and used to calculate the spine stiffness of the patient and other biomechanical data. In addition, or alternatively, the system may include a plurality of sensors, markers, or trackers on or within the vest and/or affixable to the patient to measure spine stiffness, range of motion, or other patient specific data. The system may also be configured to collect postoperative data to monitor patient outcomes. The system may record patient specific information, such as activity or pain level and other postoperative monitoring.

According to one embodiment, a system for preoperatively assessing a patient includes a wearable vest having a plurality of internal segmented compartments, a pump connected to the vest and configured to inflate the compartments of the vest, and a computer for receiving patient specific data from the vest.

The system may include one or more of the following features. The vest may include a front configured to cover an anterior side of the patient, a back configured to cover a posterior side of the patient, sides connecting the front and back configured to cover lateral sides of the patient, shoulder portions configured to rest on shoulders of the patient, and a waist section configured to rest near a waist of the patient. The vest may include a first series of compartments aligned along the front of the vest to target an anterior of the spine, a second series of compartments aligned along the back of vest to target a posterior of the spine, and third and fourth series of compartments aligned along the sides of the vest to target lateral areas of the spine. The pump may be configured to supply compressed air to inflate the compartments of the vest. The patient specific data may include biomechanical information and/or preoperative metrics of an individual patient. The biomechanical information may include spine stiffness, range of motion, and/or flexibility. A preoperative spine stiffness may be determined by an amount of force applied by the compartments of the vest and any resulting movement of the spine. The computer may simultaneously measure both spinal displacement and the force causing the displacement over an extended period of time.

According to one embodiment, a system for preoperatively assessing a spine of a patient includes a wearable vest having a plurality of segmented compartments configured to fit around a torso of the patient, the vest including a plurality of sensors configured to monitor the compartments, a pneumatic control system connected to the vest and configured to inflate the compartments of the vest, and a robotic and navigation system configured to track the plurality of sensors of the vest.

The system may include one or more of the following features. The sensors may include optical sensors, motion sensors, electromechanical sensors, or force sensors. The sensors may include infrared tracking spheres viewable by the robotic and navigation system. The system may also include a second plurality of sensors attachable to the patient, and trackable by the robotic and navigation system. The pneumatic control system may include a panel having a plurality of knobs configured to control the wearable vest. The panel may include an inlet port and a plurality of outlet ports, the inlet port being connected by pneumatic tubing to a supply of compressed air, and the outlet ports being connected to the wearable vest.

According to one embodiment, a method for preoperatively assessing a patient may include one or more of the following steps in any suitable order: (1) positioning a vest having a plurality of segmented compartments and reference sensors around a torso of the patient; (2) attaching the vest to a pneumatic control system; (3) inflating one or more compartments of the vest via the pneumatic control system to apply a force to the patient's spine; (4) monitoring the amount of force applied and any movement of the spine by a computer system; and optionally (5) attaching a plurality of sensors to the spine of the patient and monitoring any movement of the spine. The preoperative spine stiffness may be determined by the amount of force applied by the vest and any movement of the spine. When the compartments of the vest inflate to bend the patient's spine, a distance between the sensors on the patient's spine and the reference sensors on the vest change, thereby measuring displacement. The method may further include: (6) inflating one or more compartments in the vest to apply a force to an apex on a convex side of a curve of the spine; and/or (7) inflating one or more compartments in the vest to apply forces on proximal and distal ends of the curve on a concave side of the spine to create a three-point bend of the patient's spine.

According to another embodiment, a method of obtaining and measuring patient specific biomechanics may include one or more of the following steps in any suitable order: (1) positioning a wearable vest on a patient; (2) inflating chambers inside the wearable vest to apply one or more forces to the spine; (3) measuring the amount of force applied to the spine with one or more sensors, markers, or trackers on or within the vest and/or affixed to the patient; (4) determining the amount of stiffness, range of motion, flexibility, or other patient specific data; and (5) collecting, optionally analyzing, and providing data to a medical professional to provide a better understanding of the patient's specific spinal biomechanics.

Also provided are kits including biomechanical collection systems, such as wearable vests of varying types, a control panel including inlet and outlet tubing for inflating the wearable vest, and other devices and instruments for performing the procedure.

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein:

FIG. 1 shows a spinal biomechanics system including a wearable vest for obtaining patient specific data, a pump for inflating compartments within the vest, and a computer for actuating the vest and/or obtaining the patent specific data according to one embodiment;

FIGS. 2A-2C show front, side, and cross-sectional views, respectively, of a wearable vest having a plurality of segmented compartments configured to apply one or more forces to the patient to displace areas of the patient's spine according to one embodiment;

FIGS. 3A-3B show schematics of a spine having a deformity with one or more forces configured to be exerted on the spine by the vest according to one embodiment;

FIG. 4 shows an operating set-up of the wearable vest fitted on a patient with robotic or manual control of a pneumatic system for inflating the compartments of the vest according to one embodiment;

FIGS. 5A-5B show a pneumatic control apparatus system and the associated plumbing, respectively, for controlling the vest according to one embodiment;

FIG. 6 shows a rear view of a patient with sensors placed bilaterally along the spine to track displacement of the spine according to one embodiment;

FIGS. 7A-7B show front and side views, respectively, of the wearable vest with sensors configured to track the forces applied to the spine according to one embodiment;

FIGS. 8A-8B show front and side cross-sectional views, respectively, showing the vest with force sensors on or within each of the segmented compartments according to one embodiment;

FIG. 9 shows a rear view of a patient with infrared tracking spheres bilaterally placed along the spine to track displacement of the spine according to one embodiment;

FIG. 10 shows a wearable vest with infrared tracking spheres viewable and trackable by a navigation system to track the forces applied to the spine according to one embodiment;

FIGS. 11A-11B show a rear view of a patient with the infrared tracking spheres and wearing the vest with the spine shown in a deformed position and straightened position, respectively, according to one embodiment;

FIG. 12 shows a rear view of a patient wearing a vest having trackable motion sensors embedded within the vest according to one embodiment;

FIG. 13 shows a rear view of a patient wearing flexible tape with trackable motion sensors configured to assess spine stiffness and flexibility according to one embodiment;

FIG. 14 shows a workflow for postoperative monitoring, data input, and data acquisition according to one embodiment;

FIG. 15 shows a workflow for electronic health record data input according to one embodiment; and

FIG. 16 shows a workflow for electronic health record data acquisition and updates according to one embodiment.

### DETAILED DESCRIPTION OF THE INVENTION

Embodiments of the disclosure are generally directed to devices, systems, and methods for obtaining, measuring, and/or collecting preoperative and/or postoperative patient data. The preoperative patient data may include patient specific data, such as spine stiffness, range of motion, flexibility, and other biomechanical information about a specific patient. The patient specific data may include the unique health and medical information pertaining to one individual patient. The patient specific data may include demographic information, medical history, imaging, lab and test results, etc. In addition, preoperative patient data may be obtained from an individual patient in real time. Such preoperative data may include biomechanical information and preoperative metrics. Biomechanical information may include spine stiffness, range of motion, flexibility, and other biomechanical data. Preoperative metrics may include heart rate, blood pressure, body temperature, respiratory rate, oxygen saturation, level of exercise, hydration, and other pertinent medical information.

In one embodiment, the patient specific data may be obtained via an external biomechanical data collection system. The biomechanical data collection system may include a wearable device, such as a vest, which the patient may wear prior to surgery to accurately obtain and record patient specific data. The wearable vest may include a plurality of segmented compartments positioned strategically around the patient's torso. One or more compartments may be configured to apply force(s) onto the patient's spine. The force and resulting displacement to the spine may be measured and used to calculate the stiffness of the spine, range of motion, flexibility, or other biomechanical information for the patient.

In addition, or alternatively, the system may include one or more sensors, markers, or trackers located on or within the vest or other wearable patient device and/or affixable to the patient to measure spine stiffness or other patient specific data. The sensors may include, for example, optical sensors, electromechanical sensors, motion sensors, and/or force sensors. The optical sensors may include active or passive tracking markers, such as infrared tracking spheres or discs viewable by a navigation and/or robotic system. Electromechanical sensors may include accelerometers, inertial measurement units (IMU), potentiometers, or other electromechanical sensors. Motion sensors may include passive infrared sensors or active ultrasonic sensors, for example. Force sensors may include strain gages, piezoelectric sensors, and the like.

In some embodiments, the system may include postoperative patient monitoring, data input, and data acquisition, which may serve as a tool to create better preoperative plans, allow for better future intraoperative decisions, and/or improve overall patient outcomes. The biomechanical data collection system may include an interactive wearable device or software application where the patient may login to give and receive information pertaining to a recent surgery. Postoperative metrics may include surveys on pain levels, activity reports, and overall postoperative satisfaction. The system may also incorporate physical monitoring devices, such as heart monitors, pedometers, and location trackers.

Spine stiffness and range of motion are important parameters that a surgeon may consider when making a surgical plan to correct a spinal deformity. The degree of these parameters may dictate which correction maneuvers may be performed, how and where osteotomies may be needed, and what amount of correction may be expected from the surgery. Deformity correction is often easier in patients with flexible spines than it is in patients with stiff spines. Surgeons may need to perform osteotomies and/or ligament releases in patients with stiff spines in order to loosen the spine enough to achieve correction. Furthermore, the number and size of osteotomies may depend on the stiffness of the spine.

Current methods of preoperatively assessing patient spine stiffness and motion parameters may include side bending or fulcrum bending tests, push-prone tests, traction, push-traction, and flexion/extension imaging. These methods, however, rely solely on displacement and do not consider force information. In addition, most of these assessments are highly limited in the amount of data that is captured or may require a large amount of interpolation or extrapolation of the data.

To assess the true mechanical stiffness of a patient's spine, a non-invasive external system for measuring preoperative parameters may be used to simultaneously measure both spinal displacement and the force causing the displacement over a given period or extended period of time. This type of patient specific data may give the surgeon a better understanding of the patient's specific spinal biomechanics, thereby allowing the surgeon to create a better and more accurate surgical plan. The data may also be uploaded into a patient specific database or may contribute to a bio kinematic model.

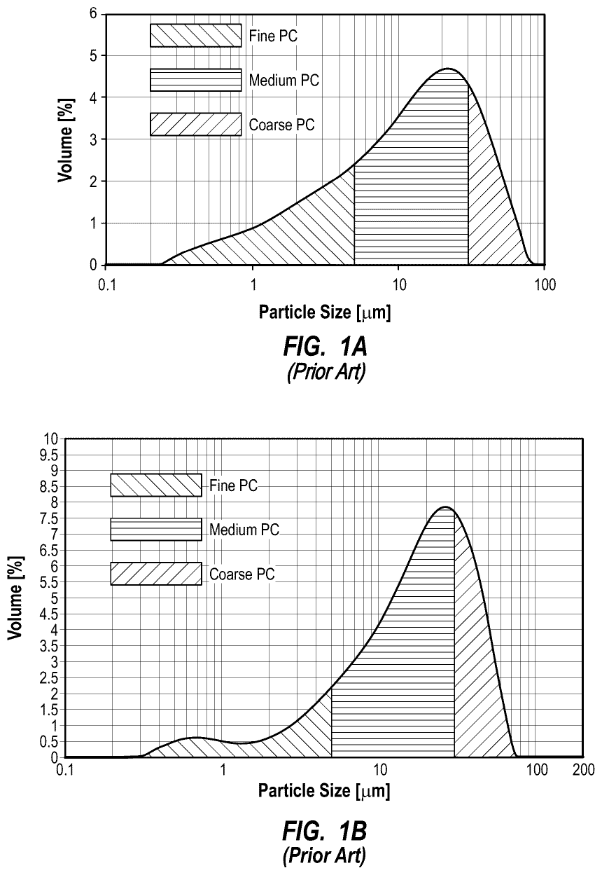

Referring now to FIG. 1, an external biomechanical data collection system 10 for preoperatively assessing the spine of a patient is shown according to one embodiment. As shown in FIG. 1, the system 10 may include a wearable vest 12, a pump 14 connected to the vest 12, and a computer 16 configured for receiving and/or measuring preoperative spine stiffness and other patient specific data. System 10 may provide the surgeon with a better understanding of the patient's specific spinal biomechanics, thereby allowing the surgeon to create a precise surgical plan to correct the spinal deformity.

The wearable vest 12 may include a vest-like device configured to cover a portion or the entire upper body or torso of the patient. The vest 12 may include a front 20 configured to cover the anterior side of the patient, a back 22 configured to cover the posterior side of the patient, and sides 24 connecting the front and back 22 configured to cover the lateral sides of the patient. The vest 12 may include shoulder portions 26 configured to rest on the shoulders of the patient and a waist section 28 configured to rest near the waist or middle of the patient.

The vest 12 includes a central open cavity 30 sized and dimensioned to receive the upper body or torso of the patient. The open cavity 30 is in fluid communication with a neck opening 32 configured to allow the patient's head to pass through comfortably, arm holes 34 defined in each side 24 configured for receiving the patient's arms therethrough, and a waist opening 36 configured to receive the patient's waist or mid-section. The front 20, back 22, and sides 24 of the vest 12 may form an integral piece such that the vest 12 may be placed over the patient's head to be worn by the patient. Alternatively, the vest 12 may define additional slits or openings configured to allow for easy placement and removal of the vest 12. If needed, the vest 12 may include buttons, zippers, or other fastening members for tightening or securing the vest 12 to the patient. Also, the vest 12 may optionally include laces, elastics, buckles, or other mechanisms to allow for size adjustments. The vest 12 may be generally rigid or may be flexible depending on its materials and construction.

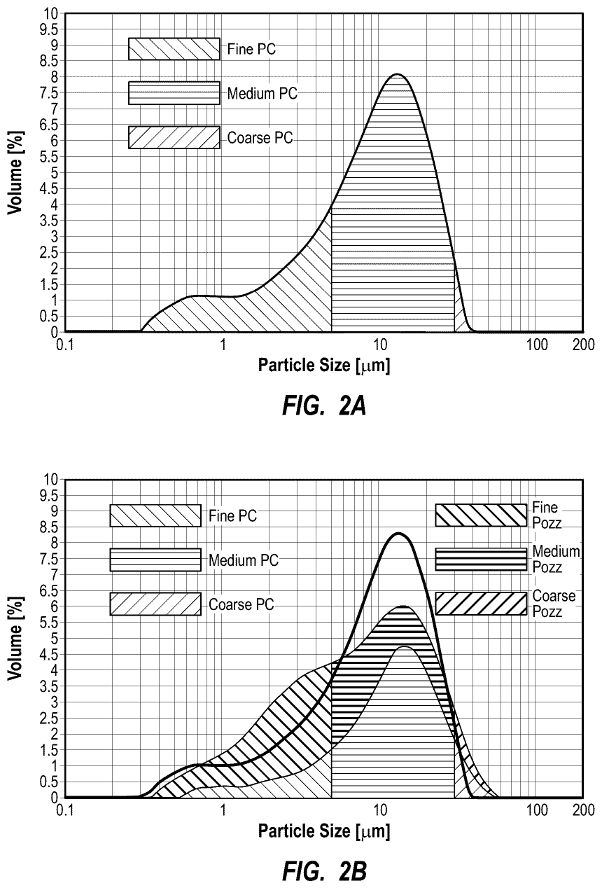

Turning now to FIGS. 2A-2C, the wearable vest 12 may include a plurality of segmented compartments 40 positioned strategically around the patient's torso. Each individual compartment 40 is configured to apply one or more forces onto the patient. In particular, each compartment 40 may be configured to apply a directed force onto the spine of the patient and may create a displacement in the patient's spine. The compartments 40 in the vest 12 may be actuated simultaneously, sequentially, individually, or in any suitable order to achieve the desired force(s).

The compartments 40 may be of any suitable size, shape, and configuration to target desired areas of the spine. For example, the compartments 40 may be arranged in a pattern configured to target specific vertebrae or areas of the spine (e.g., cervical, thoracic, lumbar, or sacral regions of the spine). In addition, the placement of the compartments 40 may be used to target anterior, posterior, or lateral aspects of the spine. In the embodiment shown, a first series of compartments 40 may be aligned along the front 20 of the vest 12 configured to target the anterior of the spine, a second series of compartments 40 may be aligned along the back 22 of the vest 12 to target the posterior of the spine, and a third and fourth series of compartments may be aligned along the sides 24 of the vest 12 to target the lateral aspects of the spine. As shown, eight individual compartments 40 aligned in two vertical columns of four may be positioned along the front 20 and back 22 of the vest 12, respectively. Six additional compartments 40 aligned in two vertical columns of three may be oriented along each of the sides 24 of the vest 12. Although a specific arrangement of compartments 40 is shown, it will be appreciated that any suitable number, type, and arrangement of compartments 40 may be provided within or on the vest 12 to impart the desired forces to the patient.

In one embodiment, the segmented compartments 40 of the vest 12 are controlled by a pneumatic system. With further emphasis on FIG. 2C, the vest 12 may have a system of lumens 42 connecting the compartments 40 within the vest 12 to the pneumatic system. For example, the vest 12 may have a series of lumens 42 connecting each of the segmented compartments 40. The lumens 42 may include internal tubes or hollow structures within the body of the vest 12. Alternatively, the lumens 42 may be external ports or attachments to each individual compartment 40 or a series of compartments 40 in the vest 12. The system of lumens 42 may be connected to pump 14, which can direct a flowable material through the lumens 42 to fill or inflate the respective compartments 40. The flowable material may be compressed air, gas, water, fluid, or other suitable material.

FIG. 2C depicts one example of the vest 12 with side compartments 40 which are inflated 40A and/or deflated 40B to impart the desired forces on the patient. Each compartment 40 is segmented and may be controllably connected or disconnected to the system of lumens 42 to control which compartments 40 are filled to exert force on the patient. The inflated compartment 40A may expand or grow in size, thereby causing the inflated compartment 40A to increase in volume and apply a pressure against the patient's body. The amount of inflation and corresponding amount of force may be monitored and controlled. The compartment 40B may be deflated by removing the compressed air, thereby causing the deflated compartment 40B to shrink back to its original size or retract to a smaller size. In FIG. 2C, the left side of the vest 12 shows upper and lower side compartments 40A inflated and middle compartment 40B deflated and the right side of the vest 12 shows upper and lower side compartments 40B deflated and middle compartment 40A inflated. The pump 14 is configured to push and pull the flowable material to and from the lumens 42 to fill and empty the respective compartments 40.

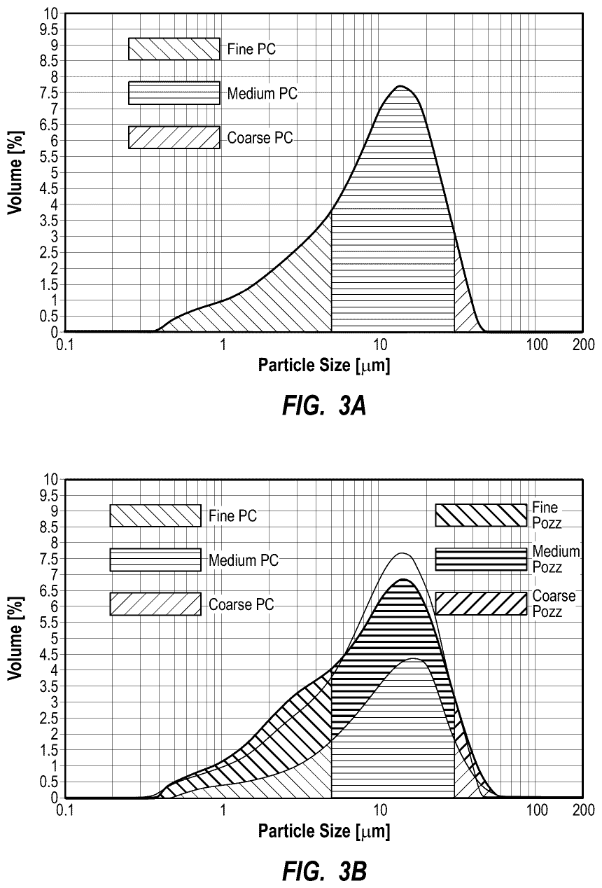

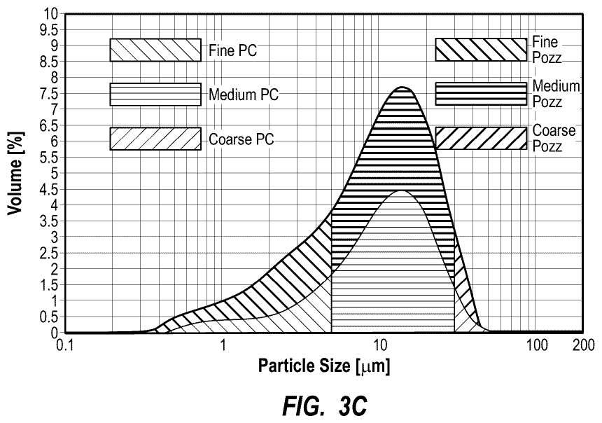

With further emphasis on FIGS. 3A-3B, the user may control which compartments 40 are inflated based on the patient's specific spinal deformity. In FIG. 3A, arrow 50 represents a force exerted at the apex on the convex side of the curve of the spine. In other words, compartments 40 positioned at the apex of the curve on the convex side of the spine may be inflated to apply force 50. When the compartments 40 on the vest 12 inflate, they may exert force 50 on the spine, thereby causing the spine to straighten. The system 10 measures the force 50 exerted on the patient and the distance the spine has moved to calculate the flexibility of the spine. In FIG. 3B, the top arrow 52 represents a force exerted at the proximal end on the concave side of the spine and bottom arrow 54 represents a force exerted at the distal end on the concave side of the spine. In this manner, the vest 12 may inflate in a configuration such that the compartments 40 on the proximal and distal ends of the curve on the concave side inflate along with those at the apex on the convex side to create a three-point bend of the patient's spine. It will be appreciated that any respective compartment(s) 40 may be inflated or deflated simultaneously, sequentially, or individually, to impart the desired forces against the patient.

In one embodiment, the compartments 40 may be inflated and/or deflated by pump 14. The pump 14 may be an air-operated pump, for example, configured to supply compressed air to the system. The pump 14 may include diaphragm pumps, piston pumps, vacuum pumps, rotary vane pumps, bellow pumps, or other suitable pumps configured to apply a suitable pressure and flow rate of air to the vest 12. The pump 14 may be connected to the vest 12 via one or more external tubes 44. The external tube 44 may be separate and distinct from the lumens 42 or the lumen system may be integral with tube 44. Although only a single tube 44 is shown in FIG. 1, it will be appreciated that any suitable number of external tubes 44 may be connected to respective adaptors or ports on the vest 12 to fill the respective compartments 40.

The vest 12 and/or pump 14 may be controlled by computer 16. The computer 16 may receive, analyze, and/or display patient specific data or information obtained from vest 12. The computer 16 may include a central processing unit (CPU), memory, and an input/output interface. The central processing unit carries out the instructions of a computer program or software by performing arithmetical, logical, control, and input/output (I/O) operations specified by these instructions. The memory may include volatile and non-volatile memory storage that temporarily or permanently store data and instructions that are currently in use or will be needed by the central processing unit. This may include, for example, random access memory (RAM), read-only memory (ROM), and storage devices like hard drives or solid-state drives. The input/output interface allows the computer system to interact with the user, take in information, and deliver results, and may include devices such as a monitor, keyboard, mouse, network interface for internet connectivity, and so forth.

The force(s) applied to the patient by compartment(s) 40 within vest 12 may create a displacement in the patient's spine. The force and/or displacement may be measured and used to calculate the spine stiffness of the patient. The spine stiffness is then recorded and displayed by the computer 16, in communication with the vest 12, for the surgeon to read and review the information. The vest 12 may be directly wired to the computer 16 or may communicate wirelessly to the computer 16. The surgeon may use system 10 to assess how stiff or flexible the patient's spine is before surgery.

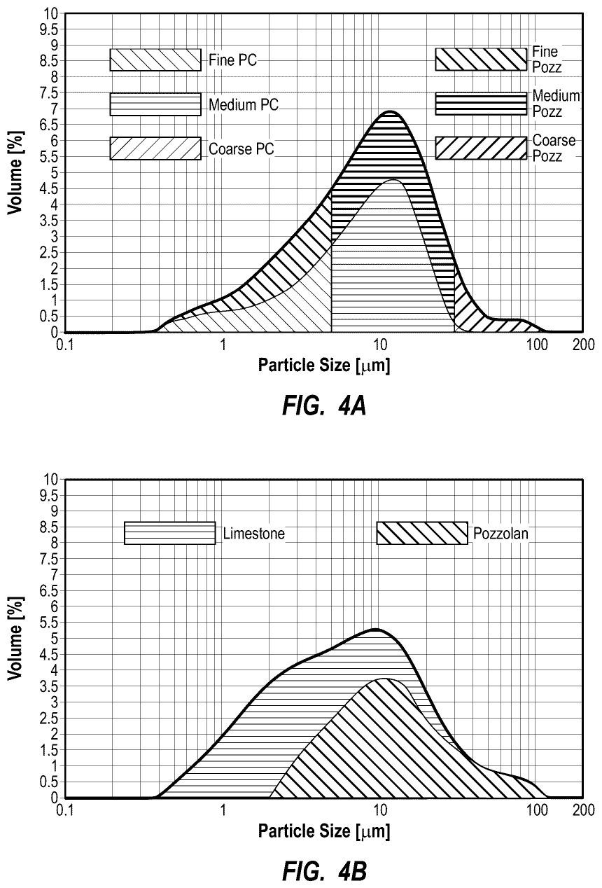

Turning now to FIG. 4, biomechanical data collection system 10 for preoperatively assessing the spine of a patient is shown in more detail according to one embodiment. The system 10 includes external vest 12 positioned on the torso of a patient 2 (the patient's extremities are omitted in the figures) and a surgical robotic and/or navigation system 60 configured to control the vest 12 and/or obtain patient specific data from the vest 12, such as preoperative spine stiffness, range of motion, flexibility, and the like.

The surgical robot and/or navigation system 60 may include, for example, surgical robot 62 having one or more robot arms 64; a base 66 on wheels containing one or more computers 16 having a processor, programming, and/or memory; a display or monitor 68 and/or optional wireless tablet (not shown) electronically or wirelessly coupled to the computer 16; an end-effector 70 including a guide tube 72 configured to receive and orient a surgical instrument, the end-effector 70 being electronically coupled to the computer 16 and movable via robot arms 64 controlled by at least one motor based on commands processed by the computer 16; and/or one or more tracking markers. The surgical robot and/or navigation system 60 may utilize a camera (not shown), for example, positioned on a camera stand to move, orient, and support the camera in a desired position. The camera may include any suitable camera, such as one or more infrared cameras (e.g., bifocal or stereophotogrammetric cameras), able to identify and track, for example, active and passive tracking markers in a given measurement volume. The system may further include 2D & 3D imaging software that allows for preoperative and intraoperative planning, navigation, and guidance. Further examples of surgical robotic and/or navigation systems can be found, for example, in U.S. Pat. Nos. 10,675,094 and 9,782,229, which are incorporated by reference herein in their entireties for all purposes.

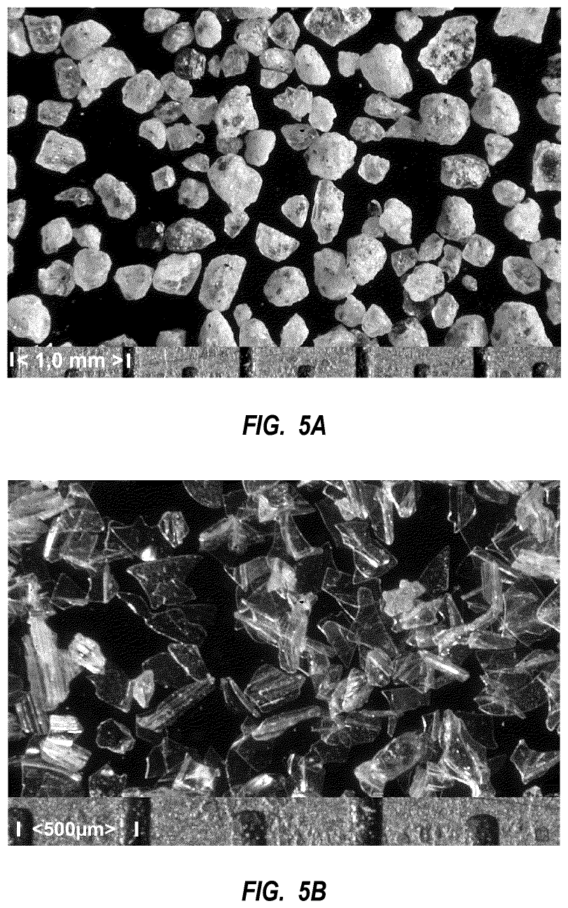

In one embodiment, the wearable vest 12 may be controlled via a pneumatic control system 80. With further emphasis on FIGS. 5A-5B, the pneumatic control system 80 may include a pneumatic distribution and control apparatus 82 and a user interface system 84 either with manual dials or knobs 86 or controllable via the robotic and/or navigated system 60. The pneumatic control apparatus 82 may include a panel 88 with user interface 84 having plurality of knobs 86 configured to be manipulated manually by the user or controlled through the robotic and/or navigated system 60. The panel 88 may include an inlet port 90 and a plurality of outlet ports 92 arranged around the side of the panel 88. The inlet port 90 may be connected by pneumatic tubing 94 to a wall outlet 96, compressor, or other suitable source and supplied with a constant supply pressure of compressed air. The outlet ports 92 may be connected by pneumatic tubing 98 to the vest 12. Although the outlet tubing 98 is consolidated into a single tube, it will be appreciated that multiple tubes 98 may be connected to the vest 12, for example, to connect to respective compartments 40 or may be otherwise configured.

FIG. 5B depicts one example of pneumatic control apparatus plumbing. A main regulator 102 may be placed in series with regional regulators 104 and segmental regulators 106, which may be connected in parallel by manifolds 108. As shown, two sets of regional regulators 104, segmental regulators 106, and manifolds 108 may be mirrored to one another. An emergency stop vent 110 may be configured to open the system to release air pressure. The emergency stop 110 may be positioned before the main regulator 102. Knobs 86 may be rotated or otherwise actuated to adjust the pressure regulator. The knobs 86 may include control knobs or valve knobs that control the flow, pressure, or direction of the compressed air within the pneumatic system.

During operation, the user may be able to open or close the valves, increase or decrease the flow rate, or adjust the pressure level according to the desired settings. Although one set-up of regulators 104, 106, 108 is shown, it will be appreciated that any suitable configuration may be used to regulate the air flow. Various configurations of the number and type of regulators, plumbing, and pressure regulators connected in series or parallel may provide more control options or simplify plumbing. It will be appreciated that the user interface 84 may be controlled manually by an operator or may be controlled by motors or other control systems. In an exemplary embodiment, the user operates the pneumatic user interface 84 via robot system 60. Although a pneumatic system with compressed air is exemplified herein, it will be appreciated that the vest and control system described herein may be modified to work with other electrical, mechanical, or alternate systems for imparting a force to the patient and/or obtaining patient specific data.

When the vest 12 applies force(s) to the patient's spine, any movement of the spine may be measured using one or more sensors, markers, or trackers. The sensors may include accelerometers, inertial measurement units (IMU), potentiometers, or other electromechanical sensors. Alternatively, movement or displacement of the spine may be measured using optical methods, such as infrared or white light tracking. The sensors, markers, or trackers may be positioned within or on the surface of the vest 12 and/or placed on the patient. The sensors or tracking markers may be positioned within the vest 12, for example, located in each compartment 40. The sensors or markers may be positioned directly on the patient, for example, attached directly to the vertebrae with a temporary fastener, to the skin of the patient, or another suitable anatomical attachment location.

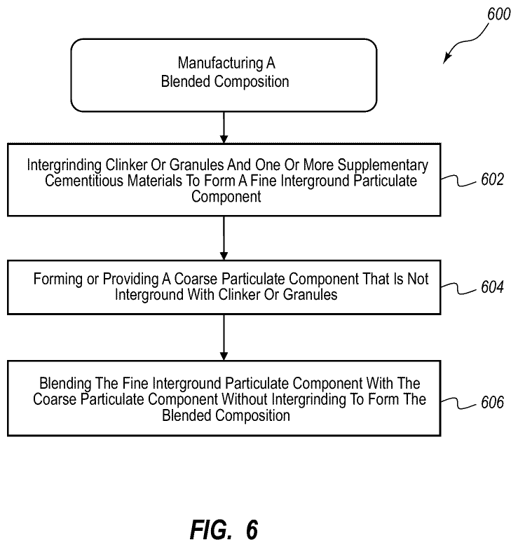

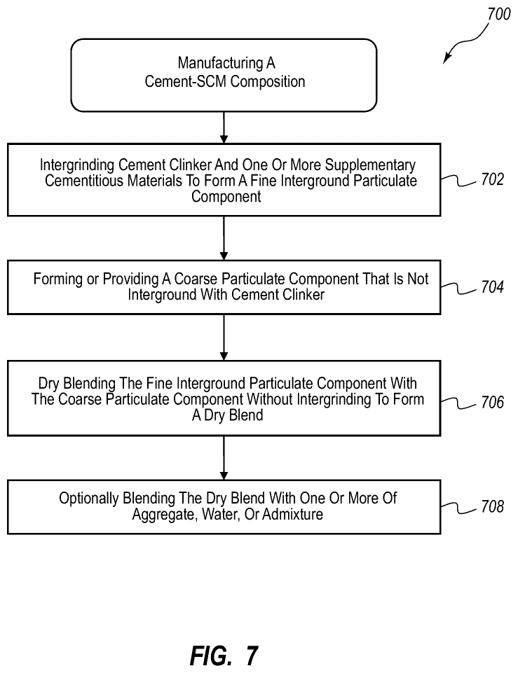

Turning now to FIGS. 6-7B, one embodiment of measuring movement of the patient's spine due to force(s) applied by the vest 12 is shown. FIG. 6 shows a posterior view of the patient 2 with a plurality of sensors 120 applied to vertebrae of the spine. For example, a temporary bone screw housing or supporting one sensor 120 may be secured bi-laterally to the pedicle of each vertebrae or selected vertebrae along the length of the spine. The sensors 120 may be independent from one another or may be linked together. FIGS. 7A-7B show anterior and lateral views of the vest 12 with a plurality of sensors 122 affixed to the vest 12. The external sensors 120 placed along the patient's spine may be optionally connected to the vest 12. Additional reference sensors 122 on the vest 12 may track the position of specific points on the vest 12. For example, the sensors 122 may be positioned within or adjacent to each compartment 40 in the vest 12. When the compartments 40 on the vest 12 inflate to bend the patient's spine, the distance between the sensors 120 on the patient's spine and the reference sensors 122 on the vest 12 change, thereby measuring displacement. The patient's spine stiffness may then be determined based on the force and displacement recorded by the system.

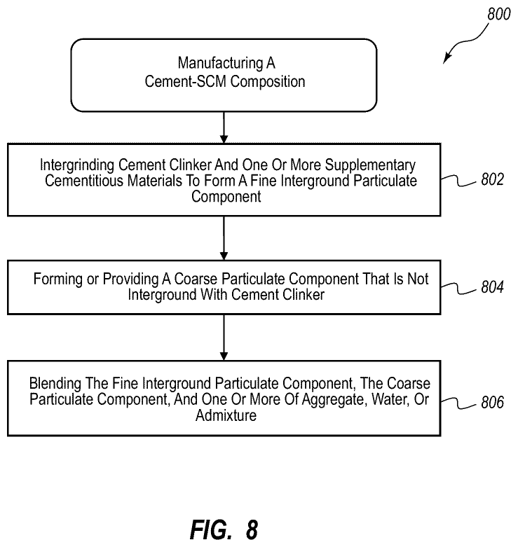

FIGS. 8A-8B show front and side cross-sectional views, respectively, of vest 12 having force sensors 124 on the segmented compartments 40 according to another embodiment. The force sensors 124 may be positioned on or within each compartment 40 of the vest 12 to measure any forces exerted on the patient as the compartment 40 inflates. A greater amount of inflation of a given compartment 40 may result in a larger amount of force exerted upon the patient. The sensors 124 measuring force may include strain gages whose measured electrical resistance varies with changes in strain, piezoelectric sensors that uses the piezoelectric effect to measure changes strain, or other types of force sensors. The force sensors 124 may be placed on the patient and/or on the vest 12 to directly measure the force(s) exerted onto the patient.

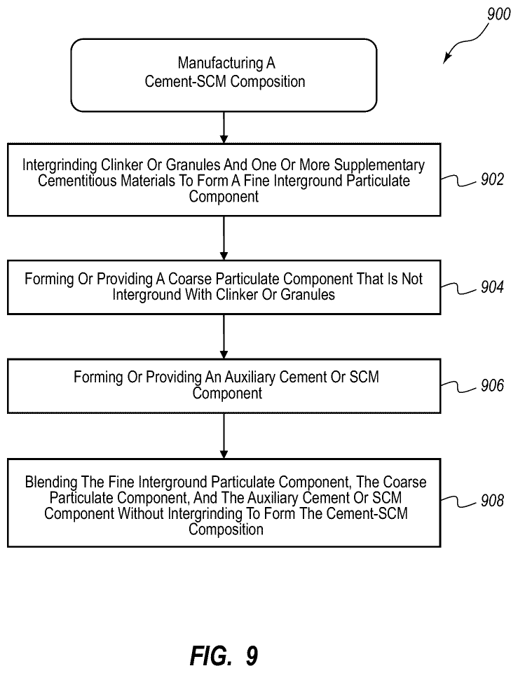

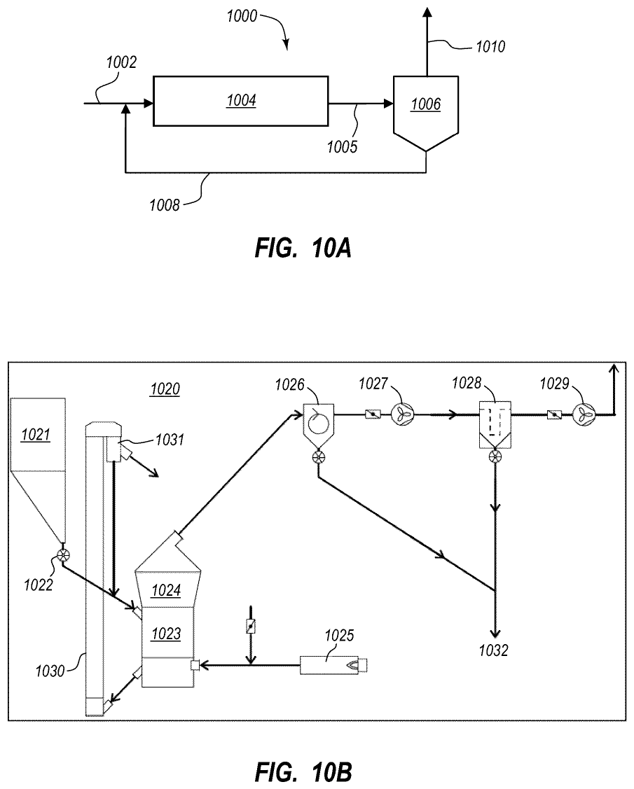

Turning now to FIGS. 9-11B, another embodiment of measuring movement of the patient's spine due to force(s) applied by the vest 12 is shown. FIG. 9 shows a posterior view of the patient 2 with a plurality of infrared tracking spheres 130 positioned along the spine. For example, a temporary bone screw supporting one or more tracking spheres 130 may be secured bi-laterally to the pedicle of each vertebrae or selected vertebrae along the length of the spine. The infrared tracking spheres may be independent from one another and viewable by the robot and navigation system 60. FIG. 10 shows an anterior view of the vest 12 with a plurality of infrared tracking spheres 132 affixed to the vest 12. The infrared tracking spheres 132 may be positioned within or adjacent to each compartment 40 in the vest 12. When the compartments 40 on the vest 12 inflate to bend the patient's spine, the distance between the infrared tracking spheres 130 on the patient's spine and the reference tracking spheres 132 on the vest 12 change, thereby measuring displacement. The system 60 may then calculate the patient's spine stiffness, range of motion, flexibility, or other biomechanics based on the force and displacement data.

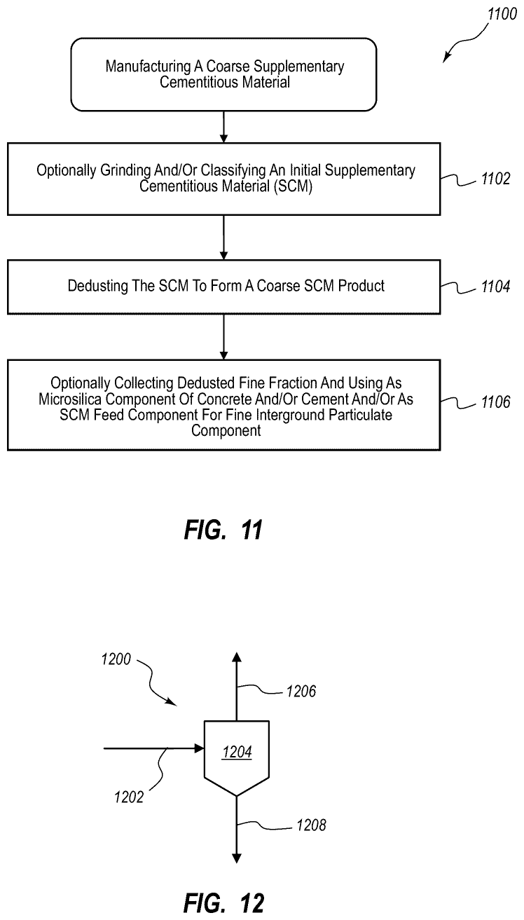

With further emphasis on FIGS. 11A-11B, the patient 2 is shown wearing the vest 12, which may be tracked by one or more fiducial markers or tracking markers 130, 132 positioned on the patient's body (shown as black spheres) and on the vest 12 (shown as white spheres). The tracking markers 130, 132 may include infrared reflective spheres viewable by the robot camera and configured to be tracked by the robotic navigation system 60. Although reflective spheres are exemplified in this embodiment, it will be appreciated that the fiducial markers may be replaced by active or passive markers of any suitable shape and type. In FIG. 11A, the spine, as marked by the black infrared tracking spheres 130 affixed to the vertebrae of the patient's spine, is shown in a deformed position. FIG. 11B shows the spine in a straightened position after force(s) are applied to the patient 2 via the vest 12. The system 60 is configured to monitor, track, and record any movement by both sets of infrared tracking spheres 130, 132. The changes to infrared tracking spheres 132 of the vest 12 correlate to forces applied to the spine and the changes to infrared tracking spheres 130 on the patient correlate to displacements of the vertebrae. The system 60 is configured to collect this patient specific data to calculate the patient's spine stiffness, range of motion, flexibility, or other biomechanics based on the patient specific data.

According to one embodiment, the system 10 and vest 12 may be automated to properly measure the patient's spine stiffness or other patient parameters. Instead of the surgeon manually choosing which compartments 40 to inflate to perform the bending test, the system 10 may be configured to receive and analyze patient data such as x-ray films, computed tomography (CT) scans, and/or magnetic resonance imaging (MRI) scans. Based on the data, the system 10 may auto-segment the patient's spine and determine where best to inflate the compartments 40 on the vest 12 to assess the spine stiffness of that patient most accurately.

According to one embodiment, a method of assessing the spine stiffness of a patient with a spinal deformity may include one or more of the following steps in any suitable order: (1) evaluating the patient for a spinal deformity, for example, by a spine surgeon; (2) taking imaging of the patient's spine, for example, x-ray, CT, or MRI scans; (3) deciding where forces should be applied to the spine in order to assess spine stiffness; (4) inputting to the system which compartments in the vest should be inflated in order to exert force in the desired locations; (5) positioning the vest on the torso of the patient; (6) pumping material, such as compressed air, into the specific compartments in the vest that were selected, for example, by the surgeon; (7) placing a force on the patient's spine due to the inflated compartments in the vest, thereby causing the spine to bend; (8) collecting, measuring, and recording force and displacement data during bending of the patient's spine; (9) calculating the patient's spine stiffness from the force and displacement data; (10) reading the resulting measure of spine stiffness, for example, by the surgeon from the system; (11) emptying the vest compartments and removing the force from the patient's spine; and (12) removing the vest from the patient.

According to one embodiment, a method of assessing the stiffness of the spine in a patient with a spinal deformity may include one or more of the following steps in any suitable order: (1) evaluating the patient for a spinal deformity, for example, by a spine surgeon; (2) taking imaging of the patient's spine, for example, via x-ray, CT, or MRI scans; (3) loading the images into the software of the system; (4) using an algorithm to automatically detect information about the patient's spinal deformity including curve type, curve magnitude, position, and orientation; (5) planning which compartments in the vest will inflate, by the computer, in order to perform a bending test on the patient's spine; (6) positioning the vest onto the torso of the patient; (7) pumping material, such as compressed air, into the specific compartments in the vest that the computer pre-planned; (8) placing a force on the patient's spine due to the inflated compartments, thereby causing the spine to bend; (9) measuring and recording force and displacement data by the computer during bending of the patient's spine; (10) calculating a measure of spine stiffness for the patient from the force and displacement data; (11) reading the measure of spine stiffness, for example, by the surgeon from the monitor of the computer; (12) emptying the vest compartments and removing the force from the patient's spine; and (13) removing the vest from the patient.

Turning now to FIG. 12, a preoperative wearable vest 112 is shown on a patient 2 according to another embodiment. Wearable vest 112 may be similar to vest 12 except the vest 112 does not include any inflatable compartments and is not configured to apply any forces to the patient 2. In this embodiment, the vest 112 is configured to obtain patient specific data, for example, on the patient's spine, range of motion, flexibility, etc. The preoperative wearable vest 112 may include one or more trackable motion sensors 140, 142, such as passive infrared sensors or active ultrasonic sensors. The sensors 140, 142 may be embedded within the vest 112 or positioned on a surface thereof. A first set of sensors 140 may be used to track the vest 112 and a second set of sensors 142 may be configured to align with the patient's spine.

The patient 2 may wear the vest 112 prior to surgery to assess the spinal range of motion or other parameters of the patient's condition more accurately. The patient specific data obtained from vest 112 may be used to create a stretching or exercise regimen to improve the patient's spinal flexibility prior to the surgery, thereby improving the patient's outcome by maximizing correction and minimizing osteotomies. These may be visible and interactive goals for the patient 2 to see and understand. The data collected from the vest 112 allows the surgeon to understand the true range of motion of the patient's spine in better detail, compared to something like flexion and extension x-rays. The use of vest 112 may replace a radiation step and create a true 3-dimensional dataset of motion to more accurately depict how the spine may react to deformity correction. It may also pinpoint specific areas to improve flexibility prior to the surgery. This data may be uploaded or used to create a patient specific or general model to simulate the potential results of correction.

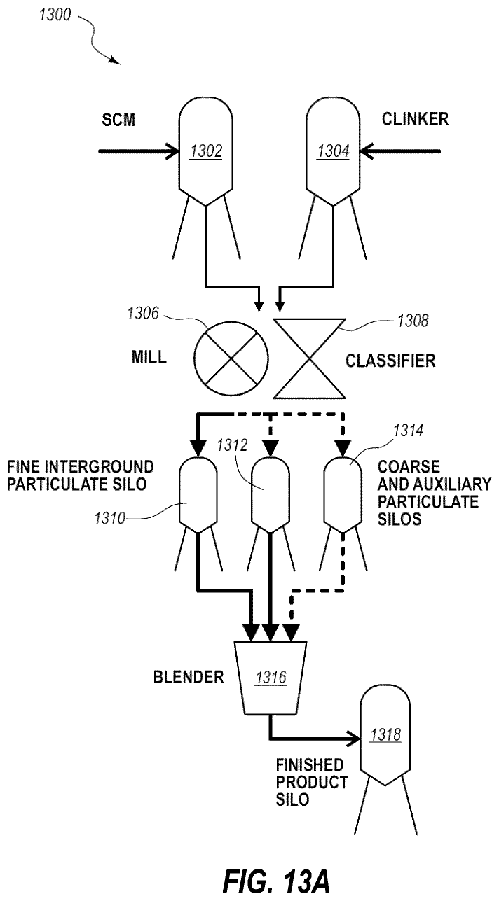

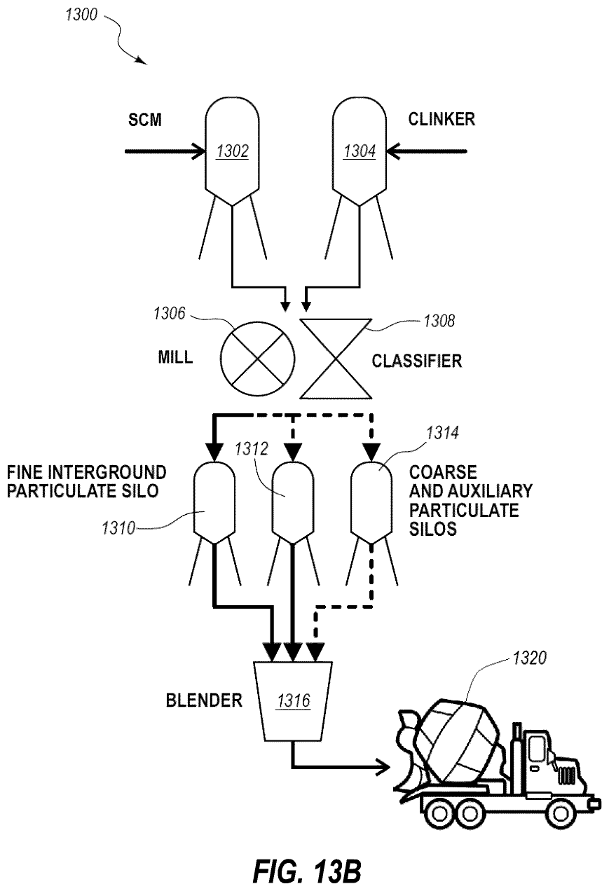

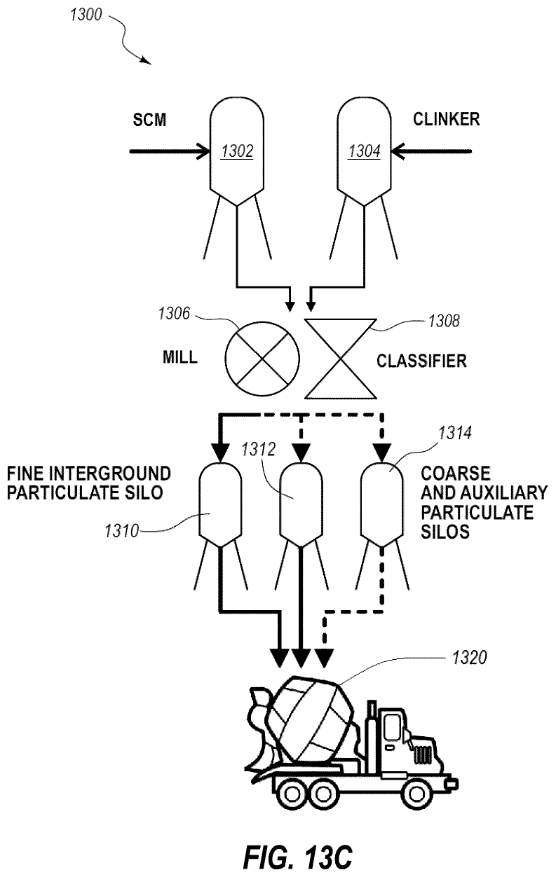

Turning now to FIG. 13, a biomechanical data collection system 160 for preoperatively assessing the spine of a patient is shown according to one embodiment. In FIG. 13, the system 160 may include flexible, wearable tape 162 applied to the patient's body 2 or other wearable device to assess spine stiffness, flexibility, or other patient parameters. The tape 162 may incorporate one or more trackable motion sensors 164, similar to sensors 140, 142. For example, sensors 164 may be embedded in or applied to each free end of the tape 162 or at other suitable locations along the tape 162. The tape 162 may be applied to the patient in specific areas of interest to target flexibility in certain directions. The tape 162 may be spread out in a grid-like pattern across the patient's back to optimize accuracy. For example, two sections of tape 162 may be aligned generally in parallel along the patient's spine and an additional two sections of tape 162 may be crisscrossed across the back. The tape 162 may be composed of a material or fabric with a known and accurate tensile spring constant, such that when certain motions are performed or a known force is applied in conjunction with the motion sensors 164, a spine stiffness number may be deduced or approximated. This may also be achieved with springs, strain gauges, or any other device linking motion, force, and/or stiffness information. Rather than sensors 164, visible light tracking may be used in conjunction with machine vision to see how the tape 162 flexes and moves to approximate range of motion.

According to another embodiment, the biomechanical data collection system may utilize one or more tracking spheres or discs, or other suitable tracking markers configured to be tracked by a navigation system (e.g., robotic and navigation system 60). The patient may come into the surgeon's office for a preoperative visit and wear the device (e.g., vest, tape, etc.) and do exercises, bends, gait analysis, etc. in front of a navigation system configured to track the markers. Force(s) may be applied to the spine while simultaneously being tracked by the navigation system, such as the force sensing actively tracked end effector 70, to obtain a spine stiffness measurement by looking at the force sensor reading within the end effector 70 and where the tracking elements on the patient's wearable move. This may also be accomplished with other methods of tracking, such as visible light, machine vision, or the like.

According to another embodiment, the biomechanical data collection system may include an interactive wearable device or software application for the patient to log and collect information pertaining to an upcoming surgery. The device may include a watch or a downloadable application, for example, on a phone or tablet. In this manner, the system functions as a communication tool between the patient and surgeon by obtaining and keeping track of preoperative patient parameters, such as heart rate, pain levels, exercise levels, hydration levels, etc. The device may request the patient's consent to access this data. The data may be used to improve a database containing patient records. The data may also be obtained from other sources, such as a phone, fitness bracelet, heart monitor, electronic health records, etc. Based on the data acquired, an anonymized patient specific file may be accessible to the patient, healthcare workers, and other interested parties.

The biomechanical data collection systems described herein allow surgeons to preoperatively assess spine stiffness, range of motion, and other patient specific biomechanics or preoperative parameters of the patient. Accurate measurement of spine stiffness, range of motion, and/or flexibility allows the surgeon to create an optimized surgical plan to correct the patient's spinal deformity based on that individual's unique spine stiffness or other properties. The wearable devices offer a more accurate and uniform method of assessing and measuring spine stiffness and flexibility than current techniques. Current techniques are often subjective and rely on patient compliance, which can lead to inaccuracies. The systems described herein create a uniform method of measurement that is independent of actions performed by the patient. The patient specific data gives the surgeon a better understanding of the patient's specific spinal biomechanics, allows the surgeon to create a better and more accurate surgical plan, and requires less time spent on decision making during the surgical procedure in the operating room. The systems may also improve communication between patient and surgeon, as well as improve the pool of patient data to further improve predictive biomechanics or other applications.

Postoperative patient data and analysis may also be helpful to understanding what affects patient outcomes. A patient may come out of a deformity spine surgery with their spine perfectly corrected to the surgeon's plan but may still be experiencing pain. Despite achieving anatomical correction, this result would still be considered a poor patient outcome if the patient's pain remains. Without postoperative assessments and check-ins, it may prove highly challenging to get this information and truly understand patient outcomes. Currently after surgery, it is up to the patient to decide whether or not to partake in rehabilitation activities or postoperative check-ins with their doctor. Accordingly, there may be more efficient and accurate ways of postoperative monitoring and analysis that may also further improve the understanding of preoperative planning and intraoperative decision making. The data may be anonymized patient data, which may be imported and exported to a patient record database. The database may be accessed and utilized during planning and/or intraoperatively for improved patient outcomes.

In one embodiment, an interactive wearable device or software application may be used for the patient to log and receive information pertaining to a recent surgery. The device may take the form of a watch or may include a downloadable application, for example, on a phone or tablet. The system may serve as a communication tool for patients and surgeons to easily interact. The patient may fill out postoperative surveys pertaining to pain levels, activity reports, and overall postoperative satisfaction. The system may also incorporate physical monitoring devices, such as heart monitors, pedometers, or location trackers. The data accumulated may be added to a patient database pool to improve other technologies pertaining to improving patient outcomes.

Turning now to FIG. 14-16, process workflows of postoperative monitoring, data input, and data acquisition are shown according to one embodiment. These processes include anonymized patient specific files that may be updated and/or accessed by the patient, surgeon, other healthcare providers, or other interested parties. In FIG. 14, process workflow 200 includes steps of entering patient data and imaging. analyzing the imaging, and saving the patient data in a system database. In step 202, a user may log into a patient file. In step 204, patient reported outcome measures (PROMs) may be entered into the system. The PROMs may be used as a tool to identify how a patient feels and is recovering postoperatively. In step 206, the PROMs data is input and recorded in the database. In step 208, test results may be entered into the system. The test results may refer to any physical or mental postoperative test/analysis deemed necessary by the healthcare provider. In step 210, the test result data is input and recorded in the database. In step 212, the system requests any imaging data. Imaging data may include but is not limited to CT scans or fluoroscopy, with the goal of analyzing postoperative spinal alignment. If available, the images are imported in step 214. In step 216, the image data is input and recorded in the database. If not available in step 218, imaging data may be obtained from previously imported data, for example. In step 220, the images are analyzed by the system. In step 222, alignment data is input and recorded in the database. In step 224, the images and alignment are displayed, for example, on a monitor. In step 226, the user verifies and saves the file.

With reference to FIG. 15, process workflow 230 of the electronic health record (EHR) data input is shown according to one embodiment. Workflow 230 is similar to workflow 200 without analyzing the imaging results. In step 202, a user may log into a patient file. In step 204, patient reported outcome measures (PROMs) may be entered into the system. In step 206, the PROMs data is input and recorded in the database. In step 208, test results may be entered into the system. In step 210, the test result data is input and recorded in the database. In step 212, the system requests any imaging data. If available, the images are imported in step 214. In step 216, the image data is input and recorded in the database. If not available in step 218, imaging data may be obtained from previously imported data, for example. In step 226, the user verifies and saves the file.

With reference to FIG. 16, process workflow 240 of the electronic health record (EHR) data acquisition for updating the patient files based on new or additional data is shown according to one embodiment. In step 242, a user or system may identify the patient files requiring an update. In step 244, the user or system may initiate an EHR pull of the relevant patient files. In step 246, the relevant data is identified. In step 248, reported patient reported outcome measures (PROMs) are identified. In step 250, the PROMs data is input and recorded in the database. In step 252, test results are identified. In step 254, the test result data is input and recorded in the database. In step 256, the system analyzes imaging data. In step 258, alignment data is input and recorded in the database. This process may be repeated for each patient file and/or each new data set. In step 260, the patient files are updated and saved. These workflows allow postoperative data to be input or retrieved directly into/from the database system.

The methods of acquiring, inputting, and applying postoperative patient data may serve as a tool to better understand what causes good patient outcomes, thereby boosting success rates of surgeries. The data may aid healthcare providers in creating better preoperative plans and making better intraoperative decisions, as well as aid developers and manufacturers in providing better tools to achieve these outcomes. These tools may also create an easier method of communication between the patient and surgeon, making it more likely for a surgeon to acquire and learn from postoperative information.

It will be further understood that various changes in the details, materials, and arrangements of the parts which have been described and illustrated in order to explain the nature of this invention may be made by those skilled in the art without departing from the scope of the invention as expressed in the claims. One skilled in the art will appreciate that the embodiments discussed above are non-limiting. It will also be appreciated that one or more features of one embodiment may be partially or fully incorporated into one or more other embodiments described herein.

What is claimed is:

1. A method for preoperatively assessing a patient, the method comprising:
positioning a vest having a plurality of segmented compartments and reference sensors around a torso of the patient;
attaching the vest to a pneumatic control system;
inflating one or more compartments of the vest via the pneumatic control system to apply a force to the patient's spine;
monitoring the amount of force applied and any movement of the spine by a computer system.

2. The method of claim 1, wherein a preoperative spine stiffness is determined by the amount of force applied by the vest and any movement of the spine.

3. The method of claim 1 further comprising attaching a plurality of sensors to the spine of the patient and monitoring any movement of the spine.

4. The method of claim 3, wherein when the compartments of the vest inflate to bend the patient's spine, a distance between the sensors on the patient's spine and the reference sensors on the vest change, thereby measuring displacement.

5. The method of claim 1 further comprising inflating one or more compartments in the vest to apply a force to an apex on a convex side of a curve of the spine.

6. The method of claim 5 further comprising inflating one or more compartments in the vest to apply forces on proximal and distal ends of the curve on a concave side of the spine to create a three-point bend of the patient's spine.

7. The system of claim 1, wherein the vest includes a front configured to cover an anterior side of the patient, a back configured to cover a posterior side of the patient, sides connecting the front and back configured to cover lateral sides of the patient, shoulder portions configured to rest on shoulders of the patient, and a waist section configured to rest near a waist of the patient.

8. The system of claim 7, wherein a first series of compartments are aligned along the front of the vest to target an anterior of the spine, a second series of compartments are aligned along the back of vest to target a posterior of the spine, and third and fourth series of compartments are aligned along the sides of the vest to target lateral areas of the spine.

9. The system of claim 1, further comprising receiving patient specific data from the vest.

10. The system of claim 9, wherein the patient specific data includes biomechanical information and/or preoperative metrics of an individual patient.

11. A method for preoperatively assessing a patient, the method comprising:
providing a system for monitoring the patient, the system including:
a wearable vest having a plurality of internal segmented compartments,
a pump connected to the vest and configured to inflate the compartments of the vest, and
a computer in electronic communication with the wearable vest; and
receiving, at the computer, patient specific data from the vest.

12. The method of claim 11, wherein a preoperative spine stiffness is determined by the amount of force applied by the vest and any movement of the spine.

13. The method of claim 11 further comprising attaching a plurality of sensors to the spine of the patient and monitoring any movement of the spine.

14. The method of claim 13, wherein when the compartments of the vest inflate to bend the patient's spine, a distance between the sensors on the patient's spine and the reference sensors on the vest change, thereby measuring displacement.

15. The method of claim 11 further comprising inflating one or more compartments in the vest to apply a force to an apex on a convex side of a curve of the spine.

16. The method of claim 15 further comprising inflating one or more compartments in the vest to apply forces on proximal and distal ends of the curve on a concave side of the spine to create a three-point bend of the patient's spine.

17. The system of claim 11, wherein the vest includes a front configured to cover an anterior side of the patient, a back configured to cover a posterior side of the patient, sides connecting the front and back configured to cover lateral sides of the patient, shoulder portions configured to rest on shoulders of the patient, and a waist section configured to rest near a waist of the patient.

18. The system of claim 17, wherein a first series of compartments are aligned along the front of the vest to target an anterior of the spine, a second series of compartments are aligned along the back of vest to target a posterior of the spine, and third and fourth series of compartments are aligned along the sides of the vest to target lateral areas of the spine.

19. The system of claim 11, wherein the patient specific data includes biomechanical information and/or preoperative metrics of an individual patient.

20. The system of claim 19, wherein the biomechanical information includes spine stiffness, range of motion, or flexibility.
